(19) United States
(12) Patent Application Publication
(10) Pub. No.: US 2015/0283346 A1
(43) Pub. Date: Oct. 8, 2015

(54) INTUMASK ASSEMBLY

(71) Applicant: BRESLAUER LTD., Petach Tikva (IL)

(72) Inventors: Haim Breslauer, Petach Tikva (IL); Shlomo Besharim, Omer (IL); Eli Besharim, Be'er - Sheva (IL)

(21) Appl. No.: 14/439,068

(22) PCT Filed: Oct. 28, 2013
(86) PCT No.: PCT/IL2013/050879

§ 371 Date: Apr. 28, 2015

Related U.S. Application Data
(60) Provisional application No. 61/719,972, filed on Oct. 30, 2012.

(51) Int. Cl. A61M 16/04; A61M 16/20; A61B 17/34; A61B 17/02; A61B 1/32; A61M 25/09; A61M 25/06

(52) U.S. Cl. CPC A61M 16/0488; A61M 16/0447; A61M 16/0427; A61M 25/09; A61M 16/044; A61M 16/20; A61M 25/0668; A61M 16/0459; A61B 17/0218; A61B 1/32; A61B 17/3423; A61M 16/0409; A61M 2205/0216; A61B 2017/0225; A61B 2017/345

(57) ABSTRACT

The present invention presents an integrated (i) inner tube, and (ii) laryngeal mask device, comprising: (i) an outer laryngeal mask tube (OLT) having proximal portion and distal portion; comprising: a balloon cuff shaped as a laryngeal mask located in said distal portion and an elongated tube in connection with the balloon cuff (TOLT); (ii) an inner tube located within said OLT characterized by a main longitudinal axis and (iii) at least one lateral movement preventing element located circumferentially to said inner tube and between said inner tube and said OLT. Thereby, when the balloon cuff is in a deflated configuration, the thickness of the same is reduced such that said balloon cuff is not protruding from the TOLT.

### FIELD OF THE INVENTION

This invention generally pertains to artificial airway assemblies. In particular, it relates to artificial airway assemblies in which the airway tube is designed for ease of function and placement, and for ensuring that the airway assembly is secured in the correct position.

### BACKGROUND OF THE INVENTION

US patent application 2003192548 discloses a laryngeal mask tube which consists of a dual-airway tube, a mask with an inflatable bladder and an inflation indicator device. The dual-airway tube comprises a simplified primary tube and secondary tube integrally combined. The primary tube communicates with the mask to guide gas into the body of a patient and the secondary tube communicates between with the bladder and the inflation indicator device to inflate the bladder and monitor the inflation. Moreover, two ribs are formed in the mask to prevent blockage of the primary tube, and a tongue is formed inside the bladder to prevent the bladder from folding and preventing a seal around the larynx. All of this ensures that the laryngeal mask airway is convenient and efficient to use.

US patent application 2004089307 discloses an artificial airway device comprising a laryngo-pharyngeal mask which includes an expandable masking ring of roughly elliptical shape. The expandable mask sealingly surrounds the laryngeal inlet when expanded, to obstruct communication between the laryngeal inlet and the esophagus. One or more airway tubes connected to the mask provide for fluid flow to a portion of the mask facing the laryngeal inlet when the mask sealingly surrounds the laryngeal inlet. A gastro-tube connected to the mask is bonded along most of its length to the airway tube and provides a fluid flow-path to the surface of the mask facing the esophagus when the mask sealingly surrounds the laryngeal inlet. The distal end of the gastro tube passes through the masking ring at its narrower distal region where, when installed in a patient, it abuts against the esophagus; and the distal portion of the gastro tube flattens when the mask is deflated to facilitate smooth passage behind the larynx during insertion through the mouth and throat of the patient.

U.S. Pat. No. 4,681,094 discloses a laryngoscope comprising a disposable blade unit including two full length blades releasably interconnected along the length thereof to define a tubular passage. A reusable handle unit removably receives one end of the blade unit and retains the blades thereof. The blade unit includes an inflatable balloon expandable against the roof of the mouth to center an inserted laryngoscope and effect movement thereof against the tongue and epiglottis. A pair of suction channels are defined through the blade unit and communicated with the interior of the tubular passage at the leading end thereof. The handle unit includes means for communicating the balloon and suction passages with sources of positive and negative air pressure respectively. The handle unit also mounts illuminating means and a selectively usable adapter for accommodating a hand-manipulable air bag for emergency respiration directly through the laryngoscope.

PCT application WO2004006746 discloses a device for the lateral separation of the air passages of patients or for single-lung ventilation during thoracic surgery. The device comprises a laryngeal mask, upstream of the larynx of the patient, in which a first tube is integrated for lung ventilation. The aim of the invention is to develop a device which enables the lateral separation of the air passages of the patient, or single-lung ventilation, in a safe and effective manner, and which enables the disadvantages of the double lumen tube used until now and the bronchus blocker available until now to be avoided. According to this invention, in addition to the laryngeal mask in which the first tube is integrated, one such device comprises a second tube which can be introduced into the air passages via the first tube.

These inventions are not guaranteed to prevent accidental insertion of the laryngeal mask into the esophagus or to prevent damage to the vocal cords. A laryngeal mask assembly that comprises active means for manipulating the tubing into the trachea still remains a long-standing but unmet need.

Furthermore, it is still a long-standing but unmet need to provide a laryngeal mask assembly without the need to detach the laryngeal mask. In other words, it would be advantageous if there were an integrated laryngeal mask assembly.

### SUMMARY OF THE INVENTION

There is provided in accordance with a preferred embodiment of the present invention an integrated (i) tubus, and (ii) laryngeal mask device, comprising: (a) an outer laryngeal mask tube (OLT) having proximal portion and distal portion; comprising: (i) a balloon cuff shaped as a laryngeal mask, having at least one inflated configuration and at least one deflated configuration, located in the distal portion; the balloon in a fluid connection with an inflating means, located at the proximal end; and, (ii) an elongated tube in connection with the balloon cuff (TOLT);

(b) an inner tube (tubus) located within the OLT; the tubus is characterized by a main longitudinal axis; and,
(c) at least one lateral movement preventing element located circumferentially to the tubus and between the tubus and the OLT; adapted both (i) to prevent lateral movement of the tubus with respect to the OLT, such that contamination of the environment between the tubus and the OLT is prevented; and (ii) to allow longitudinal reciprocating movement of the tubus along the main longitudinal axis with respect to the OLT, in a manner that a seal is provided in the inner volume between the OLT and the tubus, wherein, when the balloon cuff is in the deflated configuration, the thickness of the same is reduced such that the balloon cuff is not protruding from the TOLT.

There is further provided in accordance with a preferred embodiment of the present invention the integrated tubus and laryngeal mask device as defined above, additionally comprising a stopper adapted to fixedly secure the tubus to the operating position within the OLT.

There is further provided in accordance with a preferred embodiment of the present invention the integrated tubus and laryngeal mask device as defined above, additionally comprising a guide wire substantially accommodated along the length of the OLT; the guide wire is located in an inner canal of the laryngeal mask. There is further provided in accordance with a preferred embodiment of the present invention the integrated tubus and laryngeal mask device as defined above, wherein the guide wire is adapted for affixing and stabilizing the laryngeal mask within patient's throat.

There is further provided in accordance with a preferred embodiment of the present invention the integrated tubus and laryngeal mask device as defined above, wherein the OLT can be easily peeled off by using the guide wire.

There is further provided in accordance with a preferred embodiment of the present invention the integrated tubus and laryngeal mask device as defined above, wherein the inflating means is a pilot balloon valve connected to an inflation lumen extending along the OLT and the tubus.

There is further provided in accordance with a preferred embodiment of the present invention the integrated tubus and laryngeal mask device as defined above, wherein the lateral movement preventing element is an O-ring.

There is further provided in accordance with a preferred embodiment of the present invention the integrated tubus and laryngeal mask device as defined above, wherein the sizes of the O-ring are specified by the outside diameter of the tubus and the cross sectional diameter between the tubus and the OLT.

There is further provided in accordance with a preferred embodiment of the present invention the integrated tubus and laryngeal mask device as defined above, wherein the O-ring is an elastomeric O-ring for sealing.

There is further provided in accordance with a preferred embodiment of the present invention the integrated tubus and laryngeal mask device as defined above, wherein the lateral movement preventing element is a balloon ring.

There is further provided in accordance with a preferred embodiment of the present invention the integrated tubus and laryngeal mask device as defined above, wherein the lateral movement preventing element is a gasket which has the structure and characteristic elements enabling the lateral movement preventing element to be placed between the tubus and the OLT.

There is further provided in accordance with a preferred embodiment of the present invention the integrated tubus and laryngeal mask device as defined above, wherein the sealing structure is selected from the group consisting of: a peripheral seal, a radial seal and any combination thereof.

There is further provided in accordance with a preferred embodiment of the present invention the integrated tubus and laryngeal mask device as defined above,

wherein the stopper prevents travel of the tubus lengthwise in the OLT.

There is further provided in accordance with a preferred embodiment of the present invention the introducer as defined above, additionally comprising a maneuvering mechanism adapted to reorient the distal end of the tubus.

There is further provided in accordance with a preferred embodiment of the present invention the integrated tubus and laryngeal mask device as defined above, wherein the OLT is further adapted for guiding a working tool to an operating position to view vocal cords and trachea of the patient.

There is further provided in accordance with a preferred embodiment of the present invention the integrated tubus and laryngeal mask device as defined above, further wherein the OLT guides a working tool in a manner which allows ventilation through the tubus to continue without interruption.

There is further provided in accordance with a preferred embodiment of the present invention the integrated tubus and laryngeal mask device as defined above, additionally comprising a second inflatable balloon located at the distal end of the tubus.

There is further provided in accordance with a preferred embodiment of the present invention the integrated tubus and laryngeal mask device as defined above, additionally comprising a second valve for controlling inflation and deflation of the second balloon. There is further provided in accordance with a preferred embodiment of the present invention the integrated tubus and laryngeal mask device as defined above, wherein the second balloon is made of a flexible material.

There is further provided in accordance with a preferred embodiment of the present invention the integrated tubus and laryngeal mask device as defined above, wherein the balloon cuff is inflated to its first state when the laryngeal mask is inserted, such that it blocks entry into the esophagus of the TOLT.

There is further provided in accordance with a preferred embodiment of the present invention the integrated tubus and laryngeal mask device as defined above, wherein the first state is a state of the tubus which prevents entry of the esophagus into the internal cavity within the OLT.

There is further provided in accordance with a preferred embodiment of the present invention the integrated tubus and laryngeal mask device as defined above, wherein the second inflatable balloon is disposed about one side of, and in physical contact with, the tubus.

There is further provided in accordance with a preferred embodiment of the present invention the integrated tubus and laryngeal mask device as defined above, wherein the second balloon is in physical contact with one side of the tubus, whereby upon inflation of the second balloon while the integrated tubus and laryngeal mask device is in place, at least part of the tubus is positioned in a predetermined direction.

There is further provided in accordance with a preferred embodiment of the present invention the integrated tubus and laryngeal mask device as defined above, wherein the predetermined direction is in the direction of the trachea of the patient.

There is further provided in accordance with a preferred embodiment of the present invention the integrated tubus and laryngeal mask device as defined above, wherein the OLT has flexible walls integral with the balloon cuff of the OLT and the two are in contact when the balloon cuff is deflated.

There is further provided in accordance with a preferred embodiment of the present invention the integrated tubus and laryngeal mask device as defined above, wherein the balloon cuff is deflated to its second state once the tubus is inserted to the correct position, such that it provides no substantial obstruction within the OLT.

There is further provided in accordance with a preferred embodiment of the present invention the integrated tubus and laryngeal mask device as defined above, wherein the second state is a state in which the internal cavity within the OLT is substantially open.

There is further provided in accordance with a preferred embodiment of the present invention the integrated tubus and laryngeal mask device as defined above, wherein the balloon cuff is an expansible member made of an elastic material.

There is further provided in accordance with a preferred embodiment of the present invention the integrated tubus and laryngeal mask device as defined above, wherein the balloon cuff is attached to the OLT and communicates with an inflation lumen extending along the OLT and the tubus.

There is further provided in accordance with a preferred embodiment of the present invention the integrated tubus and laryngeal mask device as defined above, wherein the OLT is fixedly secured to the operating position of the tubus.

There is further provided in accordance with a preferred embodiment of the present invention the integrated tubus and laryngeal mask device as defined above, wherein the second inflatable balloon is disposed externally to the flexible airway.

There is further provided in accordance with a preferred embodiment of the present invention the integrated tubus and laryngeal mask device as defined above, wherein the second inflatable balloon is disposed within the flexible airway.

There is further provided in accordance with a preferred embodiment of the present invention the integrated tubus and laryngeal mask device as defined above, wherein the second inflatable balloon is integrated within the flexible airway.

There is provided in accordance with a preferred embodiment of the present invention a method for intubating a patient using an integrated tubus and laryngeal mask device, the method comprising a step of: (a) obtaining an integrated (i) tubus and, (ii) laryngeal mask device, comprising: (i) an outer laryngeal mask tube (OLT) having proximal end and distal portion; comprising: (1) a laryngeal mask with integral balloon cuff, having at least one inflated configuration and at least one deflated configuration, located in the distal portion and in fluid connection with an inflating means located at the proximal end of the OLT; and (2) a longitudinal tube in communication with the balloon cuff;

(ii) an inner tube (tubus) located within the OLT; the tubus characterized by a main longitudinal axis; and,
(iii) at least one lateral movement preventing element located circumferentially to the tubus and between the tubus and the OLT; adapted to both (i) prevent lateral movement of the tubus with respect to the OLT such that a contact-free environment between the tubus and the OLT is provided; and (ii) allow longitudinal reciprocating movement of the tubus along the main longitudinal axis with respect to the OLT; sealing the inner volume between the tube of the OLT and the tubus such that contamination of the volume is prevented;
(b) inserting the laryngeal mask in its deflated configuration into the mouth of a patient; (c) positioning the laryngeal mask adjacent to the larynx, (d) inflating the inserted balloon cuff in patient's larynx, (e) fixedly securing the tubus to the distal portion of the OLT and (f) deflating the balloon cuff, wherein, when the balloon cuff is deflated, the thickness of the same is reduced such that the balloon cuff is substantially in contact with the tube of the OLT.

There is further provided in accordance with a preferred embodiment of the present invention the method as defined above, additionally comprising a step of initiating ventilation of the patient via the proximal end.

There is further provided in accordance with a preferred embodiment of the present invention the method as defined above, additionally comprising a step of moving a working tool through the OLT.

There is further provided in accordance with a preferred embodiment of the present invention the method as defined above, additionally comprising a step of moving the working tool beyond the mask and into the trachea of the patient.

There is further provided in accordance with a preferred embodiment of the present invention the method as defined above, wherein the integrated tubus and laryngeal mask device further comprises an airway-directing mechanism disposed about one side of the tubus, the method further comprising a step of at least partially maneuvering the airway-directing mechanism whereby the tubus is directed toward the trachea of the patient.

There is further provided in accordance with a preferred embodiment of the present invention the method as defined above, additionally comprising a step of fixedly securing the OLT to the operating position of the tubus.

There is further provided in accordance with a preferred embodiment of the present invention the method as defined above, wherein the OLT is further adapted for guiding the working tool to an operating position to view vocal cords and trachea of the patient.

There is further provided in accordance with a preferred embodiment of the present invention the method as defined above, additionally comprising a step of providing a second balloon disposed about, and in physical contact with, one side of the tubus.

There is further provided in accordance with a preferred embodiment of the present invention the method as defined above, additionally comprising a step of, upon inflation of the inflatable balloon while the tubus is in place, directing at least part of the integrated tubus and laryngeal mask device in the direction of the trachea of the patient.

There is further provided in accordance with a preferred embodiment of the present invention the method as defined above, additionally comprising a step of disposing the second inflatable balloon externally to the tubus.

There is further provided in accordance with a preferred embodiment of the present invention the method as defined above, additionally comprising a step of disposing the second inflatable balloon within the flexible airway.

There is further provided in accordance with a preferred embodiment of the present invention the method as defined above, additionally comprising step of maneuvering and reorienting the distal end of the tubus.

There is further provided in accordance with a preferred embodiment of the present invention the method as defined above, additionally comprising a stopper adapted to fixedly secure the tubus in the operating position of the tubus.

There is further provided in accordance with a preferred embodiment of the present invention the method as defined above, additionally comprising a guide wire substantially accommodated along the length of the OLT; the guide wire is located in an inner canal of the laryngeal mask.

There is further provided in accordance with a preferred embodiment of the present invention the method as defined above, wherein the guide wire is adapted for affixing and stabilizing the laryngeal mask within patient's throat.

There is further provided in accordance with a preferred embodiment of the present invention the method as defined above, wherein the OLT can be easily peeled off by using the guide wire.

There is further provided in accordance with a preferred embodiment of the present invention the method as defined above, wherein the valve is a pilot balloon connected to an inflation lumen extending along the OLT and the tubus.

There is further provided in accordance with a preferred embodiment of the present invention the method as defined above, wherein the lateral movement preventing element is an O-ring.

There is further provided in accordance with a preferred embodiment of the present invention the method as defined above, wherein the O-rings has sizes specified by the outside diameter of the tubus and the cross sectional diameter between the tubus and the OLT.

There is further provided in accordance with a preferred embodiment of the present invention the method as defined above, wherein the O-ring is an elastomeric O-ring for sealing.

There is further provided in accordance with a preferred embodiment of the present invention the method as defined above, wherein the lateral movement preventing element is ring-type balloon.

There is further provided in accordance with a preferred embodiment of the present invention the method as defined above, wherein the lateral movement preventing element is any type of a gasket which has the structure and characteristics to be placed between the tubus and the OLT.

There is further provided in accordance with a preferred embodiment of the present invention the method as defined above, wherein the sealing structure is selected from the group consisting of: a peripheral seal, a radial seal, and any combination thereof.

There is further provided in accordance with a preferred embodiment of the present invention the method as defined above, wherein the stopper is adapted to prevent travel of the tubus lengthwise in the OLT.

There is further provided in accordance with a preferred embodiment of the present invention the method as defined above, wherein the stopper has size, shape and mechanism to reinforce the tubus.

There is further provided in accordance with a preferred embodiment of the present invention the method as defined above, additionally comprising a second valve for controlling inflation and deflation of the second balloon.

There is further provided in accordance with a preferred embodiment of the present invention the method as defined above, wherein the balloon cuff is made of a flexible material.

There is further provided in accordance with a preferred embodiment of the present invention the method as defined above, wherein, when inserting the tubus, the inflation of the balloon cuff to its first state blocks entry of the tubus into the esophagus.

There is further provided in accordance with a preferred embodiment of the present invention the method as defined above, wherein the first state is a state of the integrated tubus and laryngeal mask device which prevents entry of the esophagus into the internal cavity within the mask.

There is further provided in accordance with a preferred embodiment of the present invention the method as defined above, wherein the second balloon is in physical contact with one side of the tubus, whereby, upon inflation of the second balloon while the OLT is in place, at least part of the tubus is directed in a predetermined direction.

There is further provided in accordance with a preferred embodiment of the present invention the method as defined above, wherein the OLT tube has flexible walls integral with the cuff balloon of the OLT; the OLT walls and the cuff balloon walls being substantially in contact when the cuff balloon is deflated.

There is further provided in accordance with a preferred embodiment of the present invention the method as defined above, wherein the balloon is deflated to its second state once inserted to and secured in the correct position, such that it provides no substantial obstruction within the mask.

There is further provided in accordance with a preferred embodiment of the present invention the method as defined above, wherein the second state is a state in which the internal cavity within the mask is substantially open.

There is further provided in accordance with a preferred embodiment of the present invention the method as defined above, wherein the second balloon and balloon cuff are expansible members made of an elastic material.

There is provided in accordance with a preferred embodiment of the present invention a method of producing an integrated tubus and laryngeal mask device, comprising steps of: (a) providing an outer laryngeal mask tube (OLT) having proximal end and distal portion having: (i) a balloon cuff shaped as a laryngeal mask, having at least one inflated configuration and at least one deflated configuration, located in the distal portion; the balloon in a fluid connection with an inflating means, located at the proximal end, and an elongated tube in connection with the balloon cuff (TOLT); (ii) an inner tube (tubus); the tubus characterized by a main longitudinal axis; (iii) at least one lateral movement preventing element,

(b) locating the tubus within the OLT (c) positioning the lateral movement preventing element circumferentially to the tubus and between the tubus and the OLT, wherein, the balloon cuff in its deflated configuration is not protruding from the TOLT.

There is further provided in accordance with a preferred embodiment of the present invention the method as defined above, wherein the lateral movement preventing element is adapted both (i) to prevent lateral movement of the tubus with respect to the OLT, such that contamination of the environment between the tubus and the OLT is prevented; and (ii) to allow longitudinal reciprocating movement of the tubus along the main longitudinal axis with respect to the OLT, in a manner that a seal is provided for the inner volume between the OLT and the tubus.

There is further provided in accordance with a preferred embodiment of the present invention the method as defined above, additionally comprising the step of positioning a stopper at the proximal end of the OLT.

There is further provided in accordance with a preferred embodiment of the present invention the method as defined above, additionally comprising a guide substantially accommodated along the length of the OLT; the guide wire is located in an inner canal of the laryngeal mask.

There is further provided in accordance with a preferred embodiment of the present invention the method as defined above, wherein the guide wire is adapted for affixing and stabilizing the laryngeal mask within patient's throat.

There is further provided in accordance with a preferred embodiment of the present invention the method as defined above, wherein the OLT can be easily peeled off by using the guide wire.

There is further provided in accordance with a preferred embodiment of the present invention the method as defined above, wherein the tubus additionally comprises a second inflatable balloon.

There is further provided in accordance with a preferred embodiment of the present invention the method as defined above, additionally comprising the step of sealing the inner volume between the OLT and the tubus with the lateral movement preventing element.

There is further provided in accordance with a preferred embodiment of the present invention the method as defined above, wherein the seal is a peripheral seal.

There is further provided in accordance with a preferred embodiment of the present invention the method as defined above, wherein the seal is a radial seal.

There is further provided in accordance with a preferred embodiment of the present invention the method as defined above, wherein the inflating means is a pilot balloon valve connected to an inflation lumen extending along the OLT and the tubus.

There is further provided in accordance with a preferred embodiment of the present invention the method as defined above, wherein the second balloon is in fluid connection with a second inflating means located at the proximal end of the tubus.

There is further provided in accordance with a preferred embodiment of the present invention the method as defined above, wherein the second inflating means is a pilot balloon valve connected to an inflation lumen extending along the OLT and the tubus.

There is further provided in accordance with a preferred embodiment of the present invention the method as defined above, further wherein the inflating means located at the proximal end is in fluid connection with the lateral movement preventing element.

### BRIEF DESCRIPTION OF THE DRAWINGS

In order to understand the invention and to see how it may be implemented in practice, a few preferred embodiments will now be described, by way of non-limiting example only, with reference to be accompanying drawings, in which:

FIG. 1 is a cross-sectional view of a laryngeal mask assembly according to one embodiment of the invention;

FIG. 2 is a cross-sectional view of a laryngeal mask assembly in its operating position according to a second embodiment of the invention;

FIG. 3 is a cross-sectional view, of a laryngeal mask assembly in its operating position according to the embodiment of the invention illustrated in FIG. 2;

FIG. 4 illustrates a flow chart of a method for intubating a patient using a laryngeal mask assembly according to the embodiment of the invention; and

FIG. 5 illustrates a flow chart of a method for producing a laryngeal mask assembly according to the embodiment of the invention.

### DETAILED DESCRIPTION OF THE PREFERRED EMBODIMENTS

In the following description, various aspects of the invention will be described. For the purposes of explanation, specific details are set forth in order to provide a thorough understanding of the invention. It will be apparent to one skilled in the art that there are other embodiments of the invention that differ in details without affecting the essential nature thereof. Therefore the invention is not limited by that which is illustrated in the figures and described in the specification, but only as indicated in the accompanying claims, with the proper scope determined only by the broadest interpretation of the claims.

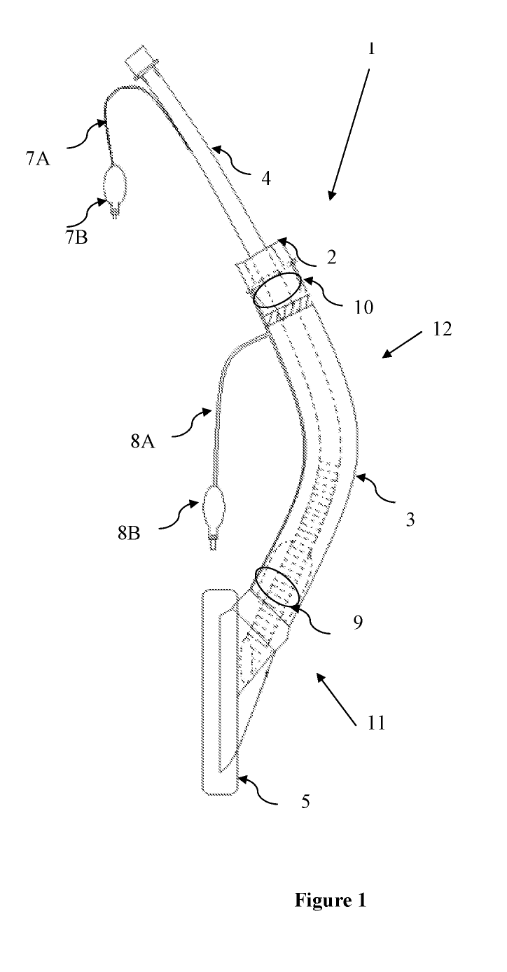

Reference is now made to FIG. 1, which shows a cross-sectional view of an artificial airway assembly before insertion according to one embodiment 1 of the present invention.

In preferred embodiments, the integrated tubus and laryngeal mask device 1 comprises an outer laryngeal mask tube (OLT) 4 having proximal portion 12 and distal portion 11. The OLT comprises a balloon cuff 5 in a communication with a longitudinal tube 3. The balloon cuff 5 is shaped as a laryngeal mask, having at least one inflated configuration and at least one deflated configuration, and is in fluid connection with an inflating means. The balloon cuff is located at the distal end 11 of the OLT. The balloon cuff is an expansible member, made of an elastic material, which has an approximately toroidal shape.

The integrated tubus and laryngeal mask device 1 further comprises a flexible airway tube (i.e., a tubus) 4 as an inner tube (tubus) located within the OLT 3. The tubus is characterized by a main longitudinal axis. The balloon cuff is connected to an inflation lumen 8A extending along the OLT and the tubus with an inflatable valve 8B, such as a pilot balloon, located at the inflation lumen edge. The valve assembly 8B is in fluid connection with the OLT and allows air to pass in and out of the OLT balloon cuff, allowing for easier insertion, as the OLT balloon cuff need not be inflated until it is already substantially in place.

The OLT is adapted to conform to the space immediately adjacent to the larynx of the patient. In preferred embodiments, the OLT is constructed of a resilient flexible biocompatible material in the form of a peripheral ring capable of defining a hollow space, capable of being inflated and deflated to accommodate the space adjacent to the larynx. When installed, the OLT adopts a configuration substantially conforming to the shape of the laryngeal inlet.

The integrated tubus and laryngeal mask device 1 further comprises at least one lateral movement preventing element 9-10 located circumferentially to the tubus and between the inner lumen of the tubus and the OLT. The lateral movement preventing element is adapted to prevent lateral movement of the tubus with respect to the OLT such that a contact-free environment between the tubus and the OLT is provided. The lateral movement preventing element is further adapted to allow longitudinal reciprocating movement of the tubus along the main longitudinal axis with respect to the OLT. The main goal of the lateral movement preventing element is to create a peripheral sealing or a radial sealing of the inner volume between the tube of the OLT and the tubus, preventing any contamination of the inner tube or infection of the throat or esophagus. The lateral movement preventing element 9-10 is selected from the group consisting of: an O ring, an inflated balloon ring or any gasket which has the structure and character to create a peripheral seal or a radial seal in between the tubus and the OLT. The O-ring acts as a mechanical gasket. It has the shape of a torus, and is designed as a loop of elastomer with a disc-shaped cross-section. The lateral movement preventing element can be positioned in the distal portion or/and in the proximal portion of the device. The lateral movement preventing element can be activated by an inflation valve assembly 7 if inflation or deflation of the element is required.

The integrated tubus and laryngeal mask device 1 further comprises a stopper 2 as an engagement part located in the proximal portion. The stopper fixedly secures the tubus to an operating position inside the OLT. The stopper prevents the tubus from traveling lengthwise in the OLT tube when ventilation is performed.

The integrated tubus and laryngeal mask device 1 further comprises a guide wire substantially accommodated along the length of the OLT. The guide wire is adapted for immutability and easy intubation of the laryngeal mask by providing a stiffness characters to the OLT. Further more, the guide wire enables the laryngeal mask to be flexible and rigid simultaneously such that it will not collapse or fold when inserted to patient's delivery system. The guide wire is positioned within an inner canal of the laryngeal mask.

The guide wire is further adapted for affixing and stabilizing the laryngeal mask within patient's throat. Further more, the OLT can be easily removed using the guide wire such that the OLT is peeled off from patient's throat whilst the tubus remains within the patient's throat.

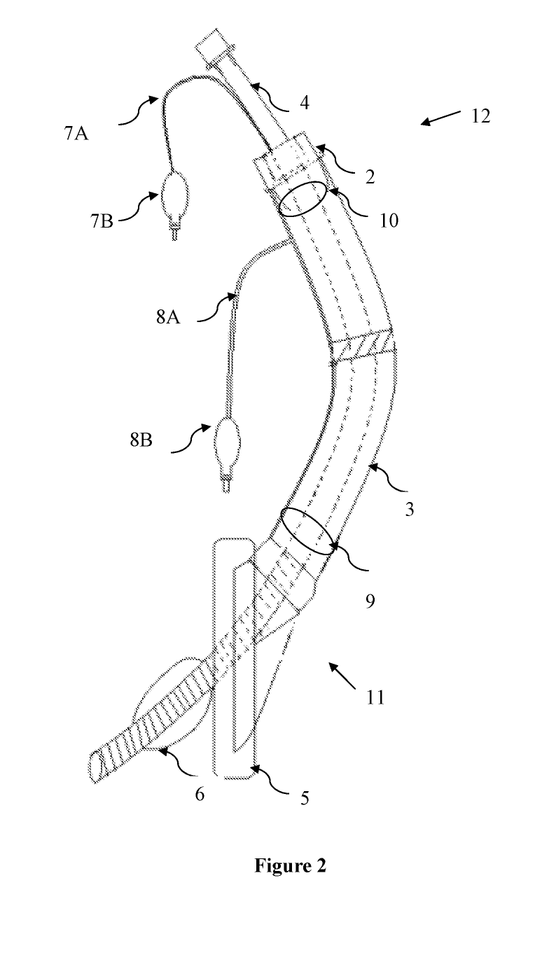

Reference is now made to FIG. 2, which shows a cross-sectional view of the integrated tubus and laryngeal mask device of a second embodiment of the invention. In this embodiment, the tubus is inserted into patient's trachea until it is substantially in place order to intubate a patient. The tubus 4 is inserted into the mouth of the patient. The OLT 5 is positioned adjacent to the larynx, thus sealing at least partially the integrated tubus and laryngeal mask device for communication exclusively through the laryngeal inlet.

In another embodiment of the invention, air is injected into the OLT balloon cuff 5 via the valve assembly 8A-8B. The distal end of the fully inflatable cuff formation prevents the more rigid distal end of the OLT from catching the inside of the throat and subjecting the patient to undesirable forces.

In another embodiment of the invention, a valve assembly 7A-7B is presented. The valve assembly 7A-7B comprises an inflation lumen 7A and an operation valve 7B located within the edge of the inflation lumen. The valve assembly is in physical connection with the tubus. The valve 7A-7B controls the inflating and deflating activation respectively of a second balloon attached to the distal end of the tubus when the integrated tubus and laryngeal mask device is substantially in place. Air can be also injected into the second balloon 6 by use of a syringe, but any means known in the art may be used instead.

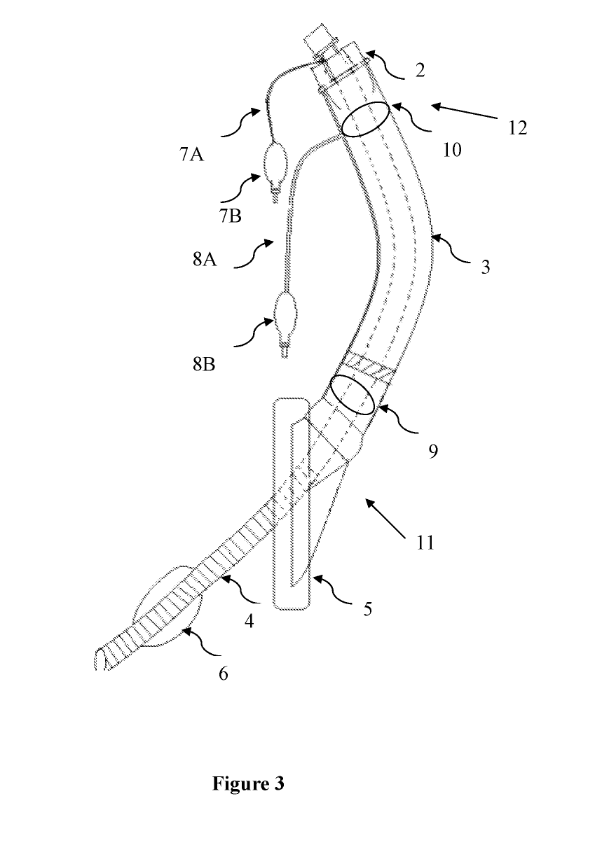

Reference is now made to FIG. 3, which shows a cross-sectional view of the integrated tubus and laryngeal mask device of a third embodiment of the invention. In this embodiment, the tubus is in its operating position as it is used to intubate a patient. The OLT balloon cuff 5 is positioned adjacent to the larynx, thus sealing at least partially the integrated tubus and laryngeal mask device for communication exclusively through the laryngeal inlet as it is positioned inside the trachea in order to start the ventilation process on a patient. When the laryngeal mask is in its insertion position, the balloon cuff 5 is inflated to its first state so that it blocks entry into the esophagus of the TOLT. Once inserted to the correct position, the balloon cuff 5 is deflated to its second state so that it provides no substantial obstruction within the mask 5.

In FIG. 3, the tubus is fixedly secured inside the patient's trachea thus allowing deflation of the balloon cuff using the valve assembly 8A-8B. When air is withdrawn and released from the inflation lumen 8A, the resilience of the balloon 5 causes it to retract. Its thickness is reduced so that the balloon cuff lies flat against the surface of the OLT and adjacent to the OLT tube's walls, resulting an integration of the cuff with the tube of the OLT. In a preferred embodiment, the OLT tube is integrated with a deflated cuff balloon (masking ring) which provides a seal around the laryngeal inlet with an adjacent independent airway supply to the patient's lungs.

In another embodiment of the invention, FIG. 3 further presents a second balloon 6 disposed about one side, which is inflated by supplying air or other fluid along the inflation lumen 7A in the usual way, so that the balloon expands to substantially fill the cavity of the trachea. The second balloon 6 is in physical contact with the tubus' distal end. The second balloon 6 may be of any appropriate biocompatible rubber-like material. In preferred embodiments of the invention, it is disposed on the outer circumference of the tubus. In other embodiments, it is disposed within tubus.

After insertion of the integrated tubus and laryngeal mask device, an additional working tool may be inserted through the proximal portion of the OLT and into the trachea in a position from which a viewing device (e.g. a fiber optic device) can be used to observe the vocal cords.

In another embodiment, the artificial airway assembly defined above is provided with a pneumatic airway-directing mechanism which comprises at least one inflatable balloon useful to direct at least part of the integrated tubus and laryngeal mask device to a predetermined location. Additionally, or alternatively, the integrated tubus and laryngeal mask device defined above, is provided with a mechanical airway-directing mechanism which comprises at least one maneuverable effecter useful to direct at least part of the flexible airway to a predetermined location. The integrated tubus and laryngeal mask device which comprises an integrated mechanism, namely a pneumatic and mechanical mechanism, is possible.

It is another object of the present invention to provide an integrated (i) tubus, and (ii) laryngeal mask device, comprising: (a) an outer laryngeal mask tube (OLT) having proximal portion and distal portion; comprising: (i) a balloon cuff shaped as a laryngeal mask, having at least one inflated configuration and at least one deflated configuration, located in the distal portion. The balloon is in a fluid connection with an inflating means, located at the proximal end; and, (ii) an elongated tube in connection with the balloon cuff (TOLT); (b) an inner tube (tubus) located within the OLT; the tubus is characterized by a main longitudinal axis; and, (c) at least one lateral movement preventing element located circumferentially to the tubus and between the tubus and the OLT; adapted both (i) to prevent lateral movement of the tubus with respect to the OLT, such that contamination of the environment between the tubus and the OLT is prevented; and (ii) to allow longitudinal reciprocating movement of the tubus along the main longitudinal axis with respect to the OLT, in a manner that a seal is provided in the inner volume between the OLT and the tubus; wherein, when the balloon cuff is in the deflated configuration, the thickness of the same is reduced such that the balloon cuff is not protruding from the TOLT.

It is another object of the present invention to provide an integrated tubus and laryngeal mask device, additionally comprising a stopper adapted to fixedly secure the tubus to the operating position within the OLT.

It is another object of the present invention to provide an integrated tubus and laryngeal mask device, additionally comprising a guide wire substantially accommodated along the length of the OLT; the guide wire is located in an inner canal of the laryngeal mask.

It is another object of the present invention to provide an integrated tubus and laryngeal mask device as defined above, wherein the inflating means is a pilot balloon valve connected to an inflation lumen extending along the OLT and the tubus.

It is another object of the present invention to provide an integrated tubus and laryngeal mask device as defined above, wherein the lateral movement preventing element is an O-ring.

It is another object of the present invention to provide an integrated tubus and laryngeal mask device as defined above, wherein the sizes of the O-rings are specified by the outside diameter of the tubus and the cross sectional diameter between the tubus and the OLT.

It is another object of the present invention to provide an integrated tubus and laryngeal mask device as defined above, wherein the O-ring is an elastomeric O-ring for sealing.

It is another object of the present invention to provide an integrated tubus and laryngeal mask device as defined above, wherein the lateral movement preventing element is a balloon ring.

It is another object of the present invention to provide an integrated tubus and laryngeal mask device as defined above, wherein the lateral movement preventing element is any type of gasket which has the structure and characteristic elements enabling the lateral movement preventing element to be placed between the tubus and the OLT.

It is another object of the present invention to provide an integrated tubus and laryngeal mask device as defined above, wherein the sealing structure is selected from the group consisting of a peripheral seal, a radial seal, and any combination thereof.

It is another object of the present invention to provide an integrated tubus and laryngeal mask device as defined above, wherein the stopper prevents travel of the tubus lengthwise in the OLT.

It is another object of the present invention to provide an integrated tubus and laryngeal mask device as defined above, additionally comprising a maneuvering mechanism adapted to reorient the distal end of the tubus.

It is another object of the present invention to provide an integrated tubus and laryngeal mask device as defined above, wherein the OLT is further adapted for guiding a working tool to an operating position to view vocal cords and trachea of the patient.

It is another object of the present invention to provide an integrated tubus and laryngeal mask device as defined above, further wherein the OLT guides a working tool in a manner which allows ventilation through the tubus to continue without interruption.

It is another object of the present invention to provide an integrated tubus and laryngeal mask device as defined above, additionally comprising a second inflatable balloon located at the distal end of the tubus.

It is another object of the present invention to provide an integrated tubus and laryngeal mask device as defined above, additionally comprising a second valve for controlling inflation and deflation of the second balloon.

It is another object of the present invention to provide an integrated tubus and laryngeal mask device as defined above, wherein the second balloon is made of a flexible material.

It is another object of the present invention to provide an integrated tubus and laryngeal mask device as defined above, wherein the balloon cuff is inflated to its first state when the laryngeal mask is inserted, such that it blocks entry into the esophagus of the TOLT.

It is another object of the present invention to provide an integrated tubus and laryngeal mask device as defined above, wherein the first state is a state of the tubus which prevents entry of the esophagus into the internal cavity within the OLT.

It is another object of the present invention to provide an integrated tubus and laryngeal mask device as defined above, wherein the inflatable balloon is disposed about one side of, and in physical contact with, the tubus.

It is another object of the present invention to provide an integrated tubus and laryngeal mask device as defined above, wherein the second balloon is in physical contact with one side of the tubus, whereby upon inflation of the second balloon while the integrated tubus and laryngeal mask device is in place, at least part of the tubus is positioned in a predetermined direction.

It is another object of the present invention to provide an integrated tubus and laryngeal mask device as defined above, wherein the predetermined direction is in the direction of the trachea of the patient.

It is another object of the present invention to provide an integrated tubus and laryngeal mask device as defined above, wherein the OLT has flexible walls integral with the balloon cuff of the OLT and the two are substantially in contact when the balloon cuff is deflated.

It is another object of the present invention to provide an integrated tubus and laryngeal mask device as defined above, wherein the balloon cuff is deflated to its second state once the tubus is inserted to the correct position, such that it provides no substantial obstruction within the OLT.

It is another object of the present invention to provide an integrated tubus and laryngeal mask device as defined above, wherein the second state is a state in which the internal cavity within the OLT is substantially open.

It is another object of the present invention to provide an integrated tubus and laryngeal mask device as defined above, wherein the balloon cuff is an expansible member made of an elastic material.

It is another object of the present invention to provide an integrated tubus and laryngeal mask device as defined above, wherein the balloon cuff is attached to the OLT and communicates with an inflation lumen extending along the OLT and the tubus.

It is another object of the present invention to provide an integrated tubus and laryngeal mask device as defined above, wherein the OLT is fixedly secured to the operating position of the tubus.

It is another object of the present invention to provide an integrated tubus and laryngeal mask device as defined above, wherein the second inflatable balloon is disposed externally to the flexible airway.

It is another object of the present invention to provide an integrated tubus and laryngeal mask device as defined above, wherein the second inflatable balloon is disposed within the flexible airway.

It is another object of the present invention to provide an integrated tubus and laryngeal mask device as defined above, wherein the second inflatable balloon is integrated within the flexible airway.

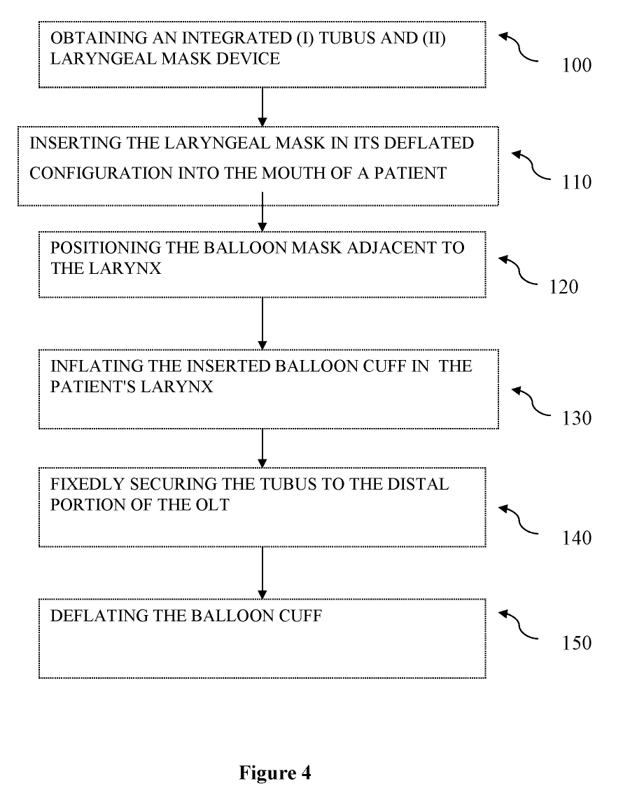

FIG. 4 illustrates a flow chart of a method for intubating a patient using a laryngeal mask assembly of the present invention. It is another object of the present invention to provide a method for intubating a patient using an integrated tubus and laryngeal mask device, the method comprising a step of: (a) obtaining an integrated (I) tubus and (II) laryngeal mask device 100, comprising: (i) an outer laryngeal mask tube (OLT) having proximal end and distal portion, comprising: (a) a laryngeal mask with integral balloon cuff, having at least one inflated configuration and at least one deflated configuration, located in the distal portion and in fluid connection with an inflating means located at the proximal end of the OLT; and, (b) a longitudinal tube in communication with the balloon cuff;

(ii) an inner tube (tubus) located within the OLT, the tubus characterized by a main longitudinal axis, and (iii) at least one lateral movement preventing element located circumferentially to the tubus and in between the tubus and the OLT; adapted to both (i) prevent lateral movement of the tubus with respect to the OLT such that a contact-free environment between the tubus and the OLT is provided, and (ii) allow longitudinal reciprocating movement of the tubus along the main longitudinal axis with respect to the OLT; sealing of the inner volume between the tube of the PLT and the tubus such that contamination of the volume is prevented;

(b) inserting the laryngeal mask in its deflated configuration into the mouth of a patient 110, (c) positioning the balloon mask adjacent to the larynx 120, (d) inflating the inserted balloon cuff in patient's larynx 130, (e) fixedly securing the tubus to the distal portion of the OLT 140; and (f) deflating the balloon cuff 150;

wherein, when balloon cuff is deflated, the thickness of the same is reduced such that the balloon cuff is substantially in contact with the tube of the OLT.

It is another object of the present to provide a method for intubating a patient using an integrated tubus and laryngeal mask device additionally comprising a step of initiating ventilation of the patient via the proximal end.

It is another object of the present to provide a method for intubating a patient using an integrated tubus and laryngeal mask device, additionally comprising a step of moving a working tool through the OLT

It is another object of the present invention to provide the method as defined above, additionally comprising a step of moving the working tool beyond the mask and into the trachea of the patient.

It is another object of the present invention to provide the method as defined above, wherein the integrated tubus and laryngeal mask device further comprises an airway-directing mechanism disposed about one side of the tubus, the method further comprising a step of at least partially maneuvering the airway-directing mechanism whereby the tubus is directed toward the trachea of the patient.

It is another object of the present invention to provide the method as defined above, additionally comprising a step of fixedly securing the OLT to the operating position of the tubus.

It is another object of the present invention to provide the method as defined above, wherein the OLT is further adapted for guiding the working tool to an operating position to view vocal cords and trachea of the patient.

It is another object of the present invention to provide the method as defined above, additionally comprising a step of providing a second balloon disposed about, and in physical contact with, one side of the tubus.

It is another object of the present invention to provide the method as defined above, additionally comprising a step of, upon inflation of the inflatable balloon while the tubus is in place, directing at least part of the integrated tubus and laryngeal mask device in the direction of the trachea of the patient.

It is another object of the present invention to provide the method as defined above, additionally comprising a step of disposing the second inflatable balloon externally to the tubus.

It is another object of the present invention to provide the method as defined above, additionally comprising a step of disposing the second inflatable balloon within the flexible airway.

It is another object of the present invention to provide the method as defined above, additionally comprising step of maneuvering and reorienting the distal end of the tubus.

It is another object of the present invention to provide the method as defined above, additionally comprising a stopper adapted to fixedly secure the tubus in its operating position.

It is another object of the present invention to provide the method as defined above, additionally comprising a guide wire substantially accommodated along the length of the OLT; the guide wire is located in an inner canal of the laryngeal mask.

It is another object of the present invention to provide the method as defined above, wherein the valve is a pilot balloon connected to an inflation lumen extending along the OLT and the tubus.

It is another object of the present invention to provide the method as defined above, wherein the lateral movement preventing element is an O-ring.

It is another object of the present invention to provide the method as defined above, wherein the O-ring has sizes specified by the outside diameter of the tubus and the cross sectional diameter between the tubus and the OLT.

It is another object of the present invention to provide the method as defined above, wherein the O-ring is an elastomeric O-ring for sealing.

It is another object of the present invention to provide the method as defined above, wherein the lateral movement preventing element is ring-type balloon.

It is another object of the present invention to provide the method as defined above, wherein the lateral movement preventing element is any type of a gasket which has the structure and characteristics to be placed between the tubus and the OLT.

It is another object of the present invention to provide the method as defined above, wherein the sealing structure is selected from the group consisting of: a peripheral seal, a radial seal, and any combination thereof.

It is another object of the present invention to provide the method as defined above, wherein the stopper adapted to prevent travel of the tubus lengthwise in the OLT.

It is another object of the present invention to provide the method as defined above, wherein the stopper has the size, shape and mechanism to reinforce the tubus.

It is another object of the present invention to provide the method as defined above, additionally comprising a second valve for controlling inflation and deflation of the second balloon.

It is another object of the present invention to provide the method as defined above, wherein the balloon cuff is made of a flexible material.

It is another object of the present invention to provide the method as defined above, wherein, when inserting the tubus, inflation of the balloon cuff to its first state blocks entry of the tubus into the esophagus.

It is another object of the present invention to provide the method as defined above, wherein the first state is a state of the integrated tubus and laryngeal mask device which prevents entry of the esophagus into the internal cavity within the mask.

It is another object of the present invention to provide the method as defined above, wherein the second balloon is in physical contact with one side of the tubus, whereby, upon inflation of the second balloon while the OLT is in place, at least part of the tubus is directed in a predetermined direction.

It is another object of the present invention to provide the method as defined above, wherein the OLT tube has flexible walls integral with the cuff balloon of the OLT and substantially in contact with them when the cuff balloon is deflated.

It is another object of the present invention to provide the method as defined above, wherein the balloon is deflated to its second state once inserted to and secured in the correct position, such that it provides no substantial obstruction within the mask.

It is another object of the present invention to provide the method as defined above, wherein the second state is a state in which the internal cavity within the mask is substantially open.

It is another object of the present invention to provide the method as defined above, wherein the second balloon and the balloon cuff are expansible members made of an elastic material.

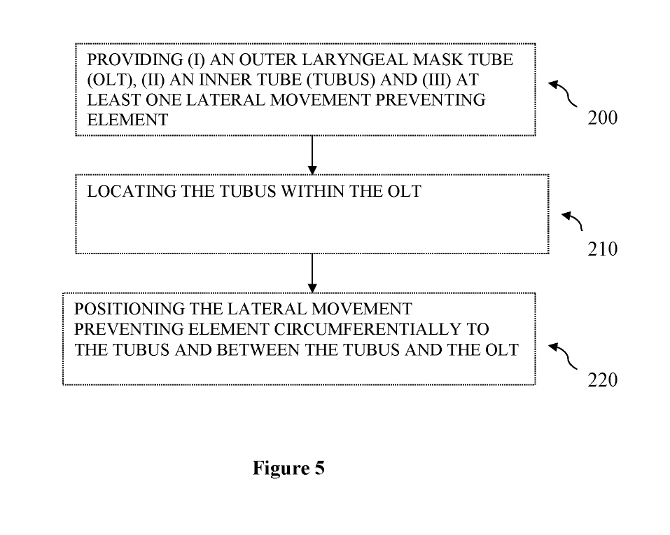

FIG. 5 illustrates a flow chart of a method for producing a laryngeal mask assembly of the present invention. It is another object of the present invention to provide a method of producing an integrated tubus and laryngeal mask device as defined above, comprising steps of: (a) providing an outer laryngeal mask tube (OLT) having proximal end and distal portion 200 having: (i) a balloon cuff shaped as a laryngeal mask, having at least one inflated configuration and at least one deflated configuration, located at the distal portion; the balloon in fluid connection with an inflating means, located at the proximal end, and an elongated tube in connection with the balloon cuff (TOLT); (ii) an inner tube (tubus); the tubus characterized by a main longitudinal axis; (iii) at least one lateral movement preventing element; (b) locating the tubus within the OLT 210, (c) positioning the lateral movement preventing element circumferentially to the tubus and in between the tubus and the OLT 220; wherein the balloon cuff in its deflated configuration is not protruding from the TOLT.

It is another object of the present invention to provide the method as defined above, wherein the lateral movement preventing element is adapted both (i) to prevent lateral movement of the tubus with respect to the OLT, such that contamination of the environment between the tubus and the OLT is prevented; and (ii) to allow longitudinal reciprocating movement of the tubus along the main longitudinal axis with respect to the OLT, in a manner that a seal is provided for the inner volume between the OLT and the tubus.

It is another object of the present invention to provide the method as defined above, additionally comprising the step of positioning a stopper at the proximal end of the OLT.

It is another object of the present invention to provide the method as defined above, wherein the tubus additionally comprises a second inflatable balloon.

It is another object of the present invention to provide the method as defined above, additionally comprising the step of sealing the inner volume between the OLT and the tubus with the lateral movement preventing element.

It is another object of the present invention to provide the method as defined above, wherein the seal is a peripheral seal.

It is another object of the present invention to provide the method as defined above, wherein the seal is a radial seal.

It is another object of the present invention to provide the method as defined above, wherein the inflating means is a pilot balloon valve connected to an inflation lumen extending along the OLT and the tubus.

It is another object of the present invention to provide the method as defined above, wherein the second balloon is in fluid connection with a second inflating means located at the proximal end of the tubus.

It is another object of the present invention to provide the method as defined above, wherein the second inflating means is a pilot balloon valve connected to an inflation lumen extending along the OLT and the tubus.

It is another object of the present invention to provide the method as defined above, further wherein the inflating means located at the proximal end is in fluid connection with the lateral movement preventing element.

1-81. (canceled)

82. An integrated (i) tubus, and (ii) laryngeal mask device, comprising:
a. an outer laryngeal mask tube (OLT) having proximal portion and distal portion; comprising:
i. a balloon cuff shaped as a laryngeal mask, having at least one inflated configuration and at least one deflated configuration, located in said distal portion; said balloon in a fluid connection with an inflating means, located at said proximal end; and,
ii. an elongated tube in connection with said balloon cuff (TOLT);
b. an inner tube (tubus) located within said OLT; said tubus is characterized by a main longitudinal axis; and,
c. at least one lateral movement preventing element located circumferentially to said tubus and between said tubus and said OLT; adapted both (i) to prevent lateral movement of said tubus with respect to said OLT, such that contamination of the environment between said tubus and said OLT is prevented; and (ii) to allow longitudinal reciprocating movement of said tubus along said main longitudinal axis with respect to said OLT, in a manner that a seal is provided in the inner volume between the OLT and the tubus
wherein, when said balloon cuff is in said deflated configuration, the thickness of the same is reduced such that said balloon cuff is not protruding from said TOLT.

83. The integrated tubus and laryngeal mask device according to claim 82, additionally comprising at least one element selected from a group consisting of (a) stopper adapted to fixedly secure said tubus to said operating position within said OLT; (b) a guide wire substantially accommodated along the length of said OLT; said guide wire is located in an inner canal of said laryngeal mask; wherein said guide wire is adapted for affixing and stabilizing said laryngeal mask within patient's throat; and any combination thereof.

84. The integrated tubus and laryngeal mask device according to claim 82, wherein at least one of the following is being held true (a) said OLT can be easily peeled off by using said guide wire; (b) said inflating means is a pilot balloon valve connected to an inflation lumen extending along said OLT and said tubus; (c) said lateral movement preventing element is a balloon ring; (d) said lateral movement preventing element is a gasket which has the structure and characteristic elements enabling said lateral movement preventing element to be placed between said tubus and said OLT; (e) said sealing structure is selected from the group consisting of: a peripheral seal, a radial seal and any combination thereof; and any combination thereof.

85. The integrated tubus and laryngeal mask device according to claim 82, wherein said lateral movement preventing element is an O-ring.

86. The integrated tubus and laryngeal mask device according to claim 85, wherein at least one of the following is being held true (a) the sizes of said O-ring are specified by the outside diameter of the tubus and the cross sectional diameter between said tubus and said OLT; (b) said O-ring is an elastomeric O-ring for sealing; and any combination thereof.

87. The integrated tubus and laryngeal mask device according to claim 83, wherein said stopper prevents travel of said tubus lengthwise in said OLT.

88. The integrated tubus and laryngeal mask device according to claim 82, additionally comprising a maneuvering mechanism adapted to reorient the distal end of said tubus.

89. The integrated tubus and laryngeal mask device according to claim 82, wherein at least one of the following is being held true (a) said OLT is further adapted for guiding a working tool to an operating position to view vocal cords and trachea of said patient; (b) said OLT guides a working tool in a manner which allows ventilation through said tubus to continue without interruption.

90. The integrated tubus and laryngeal mask device according to claim 82, additionally comprising a second inflatable balloon located at the distal end of said tubus.

91. The integrated tubus and laryngeal mask device according to claim 88, wherein at least one of the following is being held true (a) said integrated tubus and laryngeal mask device additionally comprising a second valve for controlling inflation and deflation of said second balloon; (b) said second balloon is made of a flexible material; (c) said second balloon is in physical contact with one side of said tubus, whereby upon inflation of said second balloon while said integrated tubus and laryngeal mask device is in place, at least part of said tubus is positioned in a predetermined direction; (d) said second inflatable balloon is disposed externally to said flexible airway; (e) said second inflatable balloon is disposed within said flexible airway; (f) said second inflatable balloon is integrated within said flexible airway; and any combination thereof.

92. The integrated tubus and laryngeal mask device according to claim 82, wherein said balloon cuff is inflated to its first state when the laryngeal mask is inserted, such that it blocks entry into the esophagus of the TOLT.

93. The integrated tubus and laryngeal mask device according to claim 91, wherein said first state is a state of said tubus which prevents entry of the esophagus into the internal cavity within said OLT.

94. The integrated tubus and laryngeal mask device according to claim 82, wherein said second inflatable balloon is disposed about one side of, and in physical contact with, said tubus.

95. The integrated tubus and laryngeal mask device according to claim 93, wherein said predetermined direction is in the direction of the trachea of said patient.

96. The integrated tubus and laryngeal mask device according to claim 82, wherein at least one of the following is being held true (a) said OLT has flexible walls integral with the balloon cuff of said OLT and the two are in contact when said balloon cuff is deflated; (b) said balloon cuff is an expansible member made of an elastic material; (c) said balloon cuff is attached to said OLT and communicates with an inflation lumen extending along said OLT and said tubus; (d) said OLT is fixedly secured to said operating position of said tubus; and any combination thereof.

97. The integrated tubus and laryngeal mask device according to claim 82, wherein said balloon cuff is deflated to its second state once said tubus is inserted to the correct position, such that it provides no substantial obstruction within said OLT; further wherein said second state is a state in which the internal cavity within said OLT is substantially open.

98. A method for intubating a patient using an integrated tubus and laryngeal mask device, said method comprising a step of:
a. obtaining an integrated (i) tubus and, (ii) laryngeal mask device, comprising:
i. an outer laryngeal mask tube (OLT) having proximal end and distal portion; comprising:
(1) a laryngeal mask with integral balloon cuff, having at least one inflated configuration and at least one deflated configuration, located in said distal portion and in fluid connection with an inflating means located at said proximal end of said OLT; and,
(2) a longitudinal tube in communication with said balloon cuff;
ii. an inner tube (tubus) located within said OLT; said tubus characterized by a main longitudinal axis; and,
iii. at least one lateral movement preventing element located circumferentially to said tubus and between said tubus and said OLT; adapted to both (i) prevent lateral movement of said tubus with respect to said OLT such that a contact-free environment between said tubus and said OLT is provided; and (ii) allow longitudinal reciprocating movement of said tubus along said main longitudinal axis with respect to said OLT; sealing the inner volume between said tube of said OLT and said tubus such that contamination of said volume is prevented;
b. inserting said laryngeal mask in its deflated configuration into the mouth of a patient;
c. positioning said laryngeal mask adjacent to the larynx;
d. inflating said inserted balloon cuff in patient's larynx;
e. fixedly securing said tubus to said distal portion of said OLT; and
f. deflating said balloon cuff;
wherein, when said balloon cuff is deflated, the thickness of the same is reduced such that said balloon cuff is substantially in contact with said tube of said OLT.

99. The method according to claim 98, additionally comprising at least one step selected from a group consisting of (a) initiating ventilation of the patient via the proximal end; (b) moving a working tool through said OLT; (c) moving said working tool beyond said mask and into the trachea of said patient; (d) fixedly securing said OLT to said operating position of said tubus; (e) providing a second balloon disposed about, and in physical contact with, one side of said tubus; (f) upon inflation of said inflatable balloon while said tubus is in place, directing at least part of said integrated tubus and laryngeal mask device in the direction of the trachea of said patient; (g) disposing said second inflatable balloon externally to said tubus; (h) disposing said second inflatable balloon within said flexible airway; (i) maneuvering and reorienting the distal end of said tubus; and any combination thereof.

100. The method according to claim 98, wherein at least one of the following is being held true (a) said integrated tubus and laryngeal mask device further comprises an airway-directing mechanism disposed about one side of said tubus, said method further comprising a step of at least partially maneuvering said airway-directing mechanism whereby said tubus is directed toward the trachea of said patient; (b) wherein said OLT is further adapted for guiding said working tool to an operating position to view vocal cords and trachea of said patient; and any combination thereof; (c) said integrated tubus and laryngeal mask device further comprises a stopper adapted to fixedly secure said tubus in said operating position of said tubus; (d) said integrated tubus and laryngeal mask device further comprises comprising a guide wire substantially accommodated along the length of said OLT; said guide wire is located in an inner canal of said laryngeal mask; wherein said guide wire is adapted for affixing and stabilizing said laryngeal mask within patient's throat; and any combination thereof.
